(19) United States
(12) Patent Application Publication
(10) Pub. No.: US 2008/0227734 A1
(43) Pub. Date: Sep. 18, 2008

(54) Mutant Lrp5/6 Wnt-Signaling Receptors in Cancer Diagnosis, Prognosis, and Treatment

(76) Inventors: Gunnar Westin, Uppsala (SE); Peyman Bjorklund, Uppsala (SE); Goran Akerstrom, Uppsala (SE)

Correspondence Address: DINSMORE & SHOHL, LLP, 1900 CHEMED CENTER, 255 EAST FIFTH STREET, CINCINNATI, OH, 45202, US

(21) Appl. No.: 10/580,487

(22) PCT Filed: Nov. 24, 2004
(86) PCT No.: PCT/IB04/04421

§ 371 Date: Jun. 7, 2007

Related U.S. Application Data
(60) Provisional application No. 60/524,513, filed on Nov. 24, 2003.

(51) Int. Cl. A61K 31/70; C07H 21/04; C07K 14/00; C12N 5/06; A01K 67/027; A61P 43/00; C12Q 1/68; G01N 33/574; C40B 30/06

(52) U.S. Cl. 514/44; 536/23.5; 530/350; 435/325; 435/6; 435/7.23; 506/10; 800/9

(57) ABSTRACT

A novel mutant form of lrp5 and lrp6 genes, the mutant LRP5 and LRP6 receptor proteins expressed therefrom, and a cell line which expresses the mutant LRP5 and/or LRP6 receptor proteins. Methods of diagnosing, prognosing and treating LRP5 related diseases, specifically hyperthyroidism and parathyroid tumors, and kits suitable for rapid on-site testing. Finally, methods of screening for agents capable of modulating the mutant LRP5 or LRP6 receptor proteins and pharmaceutical compositions comprising the selected agents.

### FIELD OF THE INVENTION

The present invention relates to identification and isolation of a novel gene, which is a mutant form of the lrp5 gene, its encoded mutant LRP5 receptor protein, and a parathyroid cell line expressing the mutant gene product. It further relates to the diagnosis, prognosis and treatment of various diseases, especially cancer, involving detection of the mutant gene, gene product, or downstream target proteins, to treatment of LRP5-related diseases, specifically hyperparathyroidism, parathyroid tumors, and breast tumor/cancer by inhibition of the formation or activity of the mutant LRP5 receptor protein, and to kits useful for rapid and on-site diagnosis or monitoring of certain cancerous disease states or determination of propensity to develop certain diseases.

Whereas the non-mutant lrp5 gene functions in the Wnt-signaling pathway to exhibit a particular expression pattern of downstream regulatory proteins in normal adult tissue, the mutant gene, expressed in certain tumors and disease states, yields an aberrant expression pattern of the associated regulatory proteins. Consequently, the mutant gene, its encoded protein, or cell lines comprising the mutant gene provide a diagnostic, prognostic, prophylactic and/or therapeutic target for tumors/cancer. The novel mutant gene or fragment thereof, or its encoded protein, or variants thereof, or a fragment thereof, and cell lines comprising the novel mutant gene can be used in various assays to screen for therapeutic agents.

### BACKGROUND OF THE INVENTION

Hyperparathyroidism is a disease characterized by benign tumor development in the parathyroid gland and excessive production of parathyroid hormone, which causes symptoms such as fatigue, bone pain, anxiety, irritability, and apathy. Hyperparathyroidism is a relatively common disease, affecting about 1% of the adult Swedish population, with an even higher prevalence among elderly individuals. More than 95% of patients are cured after surgery. Breast cancer is the most common malignancy affecting women in North America and Europe. Close to 200,000 cases of breast cancer were diagnosed in the United States alone in 2001. Breast cancer is the second leading cause of cancer death in American and European women behind lung cancer. The lifetime risk of any particular woman getting breast cancer is about 1 in 8 although the lifetime risk of dying from breast cancer is much lower at 1 in 28. The earlier that a breast cancer is found, the more likely it is that treatment can be curable. Understanding the molecular and genetic bases of parathyroid and breast tumor development will provide targets for medical treatment or prevention of parathyroid tumors, hyperparathyroidism and breast tumor/cancer.

The regulation of cell growth and survival can be subverted by a variety of genetic defects that alter transcriptional programs normally responsible for controlling cell number. Dysregulation of the Wnt-signaling pathway by stabilization of the cell-cell adhesion protein, β-catenin, with resultant accumulation of constitutive β-catenin, a transcriptional activator, is common to many human cancers (see, e.g., Lustig, B. & Behrens, J. “The Wnt-signaling pathway and its role in tumor development,” J. Cancer Res. Clin. Oncol. 129, 199-221, 2003). Mutated regulatory genes in the Wnt-signaling pathway are known to promote experimental cancers in animal subjects and the common denominator of the activation is activation of gene transcription by β-catenin.

The stability of β-catenin is regulated by Wnt-signaling through a “destruction complex” consisting of APC/Axin/GSK-3β/Dvl and other known factors. In the absence of Wnt, free cytoplasmic β-catenin is rapidly degraded. When cells are exposed to Wnt, it binds to the cell surface “Frizzled” receptors and LRP 5/6 co-receptors. According to a current model the destruction complex is then recruited to the intracellular domain of LRP5 through axin. See, e.g. Mao, J. et al. “LRP5 binds to axin and thereby regulates the canonical Wnt-signaling pathway,” Mol. Cell 7, 801-809 (2001). This results in Axin dephosphorylation and degradation with subsequent accumulation of nonphosphorylated β-catenin. β-catenin binds the LEF/TCF family of transcription factors to positively or negatively regulate transcription of target genes.

Many mutant proteins of the Wnt-signaling pathway, such as β-catenin, APC, axin, and β-Trcp, are already known to be associated with specific forms of cancer. For example, atypical accumulation of β-catenin due to mutations which stabilize β-catenin or inactivate APC is strongly implicated in the cause of approximately 10% and 80% of colorectal cancers, respectively, see Giles, R. H., van Es, J. H. & Clevers, H. “Caught in a Wnt storm: Wnt-signaling in cancer,” Biochem. Biophys. Acta 1653, 1-24 (2003). However, approaches which focus on the study of mutations in genes encoding Wnt ligands or receptors, which should provide more specific intervention sites, are scarce. Currently there is a lack of therapeutic agents available which act upstream from β-catenin to effectively inhibit its transcriptional activation.

### SUMMARY OF THE INVENTION

LRP5/6 receptors provide attractive, novel targets for the development of a new class of anti-cancer drugs which specifically inactivate the mutated constitutively active receptor while leaving the normal protein unaffected.

It has been found that the Wnt co-receptors LRP5 and LRP6 are important components to Wnt-signaling-mediated tumorogenesis. Certain tumors are known to exhibit an aberrant profile of Wnt-signaling target protein accumulation. The present inventors surprisingly discovered that a mutant lrp5 nucleotide sequence and the encoded mutant LRP5 receptor protein product is expressed at high levels in certain disease states as well as in certain tumors and cancers, in particular tumors of the parathyroid and breast, which correlates with the aberrant target protein profile. In particular, the present invention relates to the detection of these mutant receptors in various disease states and cancers, specifically in conditions and tumors/cancers related to the parathyroid and breast. The present invention encompasses therapeutic, prognostic and diagnostic applications based on the mutant lrp5 gene or mutant LRP5 receptor protein product expressed therefrom, and treatment, inhibition or prevention of tumorogenesis based on agonist or antagonist ligands for the receptor or transcriptional inhibitors. The present invention further encompasses screening assays to identify modulators of LRP5 activity and/or expression as potential therapeutic agents for the treatment, inhibition and/or prevention of certain disease states or tumorigenesis, and diagnostic kits based on the related technology.

Accordingly, one embodiment of the invention provides an isolated nucleic acid molecule which has at least 90% homology with the sequence of nucleotides as set forth in SEQ ID NO: 1. Another embodiment is directed to a cell line comprising the molecule. Another embodiment provides an isolated nucleic acid molecule encoding a polypeptide comprising a mutant LRP5 receptor protein, the molecule comprising an in-frame deletion of base pairs which encode a third YWTD β-propeller domain of an LRP5 receptor protein. A further embodiment provides an isolated polypeptide comprising an LRP5 receptor having a mutation wherein the mutation comprises a deletion of a third YWTD β-propeller domain.

Several additional embodiments are directed to methods relating to the mutant lrp5 gene and/or the expressed LRP5 receptor. One such embodiment provides a method for diagnosing, prognosing, or determining the risk of developing an LRP5-related disease. The method comprises: a) providing a tissue sample from a patient; b) detecting in the sample a mutant lrp5 gene or a mutant LRP5 receptor protein encoded by the mutant lrp5 gene; and c) relating presence of the mutant lrp5 gene or the mutant LRP5 receptor protein to an LRP5-related disease. Additional embodiments of the invention are directed to methods wherein the detection step involves noting the binding activity of the receptor, or noting the presence or absence of target proteins downstream from the LRP5 receptor in the Wnt-signaling pathway.

Another embodiment provides a method of screening agents for an ability to modulate mutant LRP5 receptor activity. The method comprises: a) generating a cell line which expresses a mutant LRP5 receptor; b) optionally, isolating the mutant LRP5 receptor from the cell line; c) pre-plating at least one plate with one or more agents; d) plating the at least one plate with cells from a), or with isolated mutant LRP5 receptors from b); e) incubating the at least one plate for a suitable period of time; and f) analyzing the at least one plate to determine if the one or more agents modulate mutant LRP5 receptor activity. A further embodiment includes additional method steps designed to screen the agent determined to modulate mutant LRP5 receptor activity for an ability to modulate non-mutant LRP5. These comprise: a) providing a second cell line which does not express the mutant LRP5 and expresses a non-mutant LRP5 receptor; b) optionally, isolating the non-mutant LRP5 receptor from the cell line; c) pre-plating at least one plate with one or more of the agents determined to modulate mutant LRP5 receptor activity; d) plating the at least one plate with cells from a), or with isolated non-mutant LRP5 receptors from b); e) incubating the at least one plate for a suitable period of time; and f) analyzing the at least one plate to determine if the one or more agents modulate non-mutant LRP5 receptor activity and identifying any remaining agent as a selected agent. In one embodiment the ability to modulate mutant LRP5 receptor activity is at a transcriptional level and the at least one agent is a small interfering RNA (siRNA). Another embodiment provides a pharmaceutical composition which comprises an agent that is selected according to these methods, along with a pharmaceutically acceptable vehicle.

An additional embodiment provides a method for identifying a ligand which modulates mutant LRP5 receptor activity. The method comprises: a) contacting a polypeptide comprising the amino acid sequence set forth as SEQ ID NO:5, or a ligand-binding fragment thereof, with at least one ligand; and b) determining binding activity of the at least one ligand with respect to the polypeptide.

A further method embodiment is directed to determining the therapeutic effectiveness of a tumor/cancer treatment. The method comprises: a) providing tumor/cancer cells; b) determining mutant LRP5 receptor activity in the tumor/cancer cells; c) providing treated tumor/cancer cells; d) determining mutant LRP5 receptor activity in the treated tumor/cancer cells; e) comparing b) to d) wherein a decrease in mutant LRP5 receptor activity in d) relative to b) indicates the treatment is therapeutically effective.

A further embodiment provides a transgenic non-human animal having a genome comprising the nucleic acid molecule having at least 90% homology to the nucleotide sequence set forth in SEQ ID NO: 1. An additional embodiment is directed to a kit for diagnosing or prognosing a disease characterized by the expression of a mutant LRP5 receptor in a tissue, comprising: a) one or more reagents having specificity for a mutant lrp5 gene or a mutant LRP5 receptor expressed therefrom, wherein the one or more reagents emits a detectable signal in the presence of the mutant lrp5 gene or the mutant LRP5 receptor expressed therefrom which is different from that emitted in the absence of the mutant lrp5 gene or the mutant LRP5 receptor expressed therefrom; b) means to deliver the one or more reagents to the tissue; and c) means suitable to detect the detectable signal.

### BRIEF DESCRIPTION OF THE DRAWINGS

FIGS. 1a-1c: illustrate aberrant β-catenin expression in parathyroid tumors.

FIGS. 2a-2e: illustrate an in-frame deletion of LRP5 detected in parathyroid tumor DNA and cDNA.

FIG. 3a-3f: illustrate β-catenin accumulation and target gene transcription in mutant LRP5 expressing cells.

FIG. 4: illustrates blocked accumulation of β-catenin in the sHPT cell line by transfection of siRNA against LRP5Δ666-809.

### DETAILED DESCRIPTION OF THE INVENTION

Unless otherwise defined, all technical and scientific terms used herein have the same meaning as commonly understood by one of ordinary skill in the art to which this invention pertains. Exemplar methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting. Other features, objects, and advantages of the invention will be apparent from the description and the accompanying drawings, and from the claims.

As used herein:

The phrase” % homology” refers to the percentage of sequence similarity found in homologues of a particular amino acid or nucleic acid sequence when comparing two or more of the amino acid or nucleic acid sequences.

The modifier “substantially identical” means greater than or equal to 95% homology between nucleotide sequences and greater than or equal to 98% identity between amino acid sequences

The term “expression,” refers to the transcription and stable accumulation of sense (mRNA) or antisense RNA derived from the nucleic acid fragment of the invention. Expression may also refer to translation of mRNA into a polypeptide.

The term “vector” refers to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequences into a cell.

The phrase “RNA transcript” refers to the product resulting from RNA polymerase-catalyzed transcription of a DNA sequence. “Messenger RNA” or “mRNA” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to an RNA transcript that is complementary to all or part of a target primary transcript or mRNA and that blocks the expression of a target gene.

The term “antibody” as used herein encompasses both monoclonal and polyclonal antibodies that fall within any antibody class, or derivatives thereof. The term “antibody” also includes antibody fragments including conjugates of such fragments, and single-chain antibodies comprising an antigen recognition epitope. In addition, the term “antibody” also means humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

The term “gene” refers to a nucleic acid fragment that expresses a specific protein, including regulatory sequences preceding (5′ non-coding sequences) and following (3′ non-coding sequences) the coding sequence. “Non-mutant” gene refers to a gene as found in nature which does not comprise the in-frame deletion mutation of the novel mutant LRP5 gene as disclosed herein.

The term “isolated” when used in reference to nucleic acids (which include gene sequences) of this invention is intended to mean that a nucleic acid molecule is present in a form other than that found in nature.

The term “domain” means a functional portion, segment or region of a protein, or polypeptide.

The term “transgenic” to describe an organism refers to the situation wherein genetic material has been introduced into the genome of the organism by a transformation procedure.

The term “complementary” is used to describe the relationship between nucleotide bases that are capable of hybridizing to one another. Accordingly, the instant invention also includes isolated nucleic acid fragments that are complementary to the complete sequences as reported in the accompanying Sequence Listing, as well as those substantially identical nucleic acid sequences.

The term “activity” when used in connection with proteins or protein complexes means any physiological or biochemical activities displayed by or associated with a particular protein or protein complex including but not limited to activities exhibited in biological processes and cellular functions, ability to interact with or bind another molecule or a moiety thereof, binding affinity or specificity to certain molecules, in vitro or in vivo stability (e.g., protein degradation rate, or in the case of protein complexes, the ability to maintain the form of a protein complex), antigenicity and immunogenicity, enzymatic activities, etc. Such activities may be detected or assayed by any of a variety of suitable methods as will be apparent to any person of ordinary skill in the art.

Low density lipoprotein receptor-related proteins 5 and 6 (LRP5 and LRP6) are Wnt coreceptors in the canonical signaling pathway. The Wnt family of secreted signaling molecules is essential in embryonic induction, cell polarity generation and cell fate specification. Deregulation of Wnt-signaling results in defects in development and growth control. The canonical Wnt pathway involves activation of β-catenin-dependent transcription and is highly evolutionarily conserved. Mutations in components, which constitutively activate canonical signaling, have been identified in several tumor types, including prostate and colorectal cancer.

Wnt binds to two coreceptors, the Frizzled-type seven transmembrane-domain receptor, and the low-density receptor-related protein (LRP) 5/6 in vertebrates. These interactions cause β-catenin stabilization through inhibition of its phosphorylation by glycogen synthase kinase 3β (GSK3β), which is assembled in a large cytoplasmic complex that includes, inter alia, Axin, a key scaffolding protein which tethers β-catenin to GSK3β for phosphorylation and degradation. As a consequence, stabilized cytoplasmic β-catenin is translocated to the nucleus and forms a complex with a family of high-mobility group-like transcription factors, including leukocyte enhancer factor-1 and T-cell factors, activating transcription of target genes. Without being bound by theory, in the most current model of the mechanism involved in Wnt-signaling through LRP, Axin is thought to bind to the LRP5 cytoplasmic domain. The extracellular domain exerts an inhibitory effect on signaling through this receptor.

LRP5 and LRP6 receptors specifically function in the canonical pathway. Biochemical interaction studies support a dual-receptor model in which independent binding to both frizzled and LRP5/6 by Wnts recruits these two types of receptors into a complex and elicits signaling to downstream components.

The present inventors discovered β-catenin accumulation in 100% of the analyzed parathyroid tumors taken from patients with hyperparathyroidism (HPT) and an in-frame deletion of the Wnt co-receptor low-density lipoprotein receptor-related protein 5 (LRP5) in 87% of these tumors. Accordingly, expression of a shorter LRP5 transcript is found in the parathyroid tumor cells. The deletion (Δ666-809) includes the third YWTD β-propeller domain of the LRP5 receptor protein. Functional studies in a cell culture of LRP5Δ666-809 reveal stabilization of β-catenin, constitutive activation of the endogenously expressed c-myc proto-oncogene, and simultaneous association of β-catenin to the c-myc promoter. C-myc is known to play a role in normal parathyroid cell cycle regulation and overexpression conceivably contributes to the enlarged overactive parathyroid glands characteristic of HPT. The majority of parathyroid tumors are found to overexpress c-myc as well. Silencing of endogenous mutant LRP5 receptor expression in parathyroid cells abolishes β-catenin accumulation. Expression of the mutant LRP5 receptor is detected in breast tumors as well. These findings suggested an important role for LRP5 in Wnt-signaling-mediated tumorigenesis and demonstrate a fundamental role of β-catenin in parathyroid and breast tumors.

Accordingly, one embodiment of the present invention is directed to an isolated nucleic acid molecule having at least 90% homology with the sequence of nucleotides as set forth in SEQ ID NO: 1. A more specific embodiment is directed to an isolated nucleic acid molecule comprising a sequence of nucleotides substantially identical to that set forth in SEQ ID NO: 1.

In a further embodiment, an isolated nucleic acid molecule is provided which encodes a polypeptide comprising a mutant low density lipoprotein related protein 5 (LRP5) or 6 (LRP6), the molecule comprising an in-frame deletion of base pairs encoding a third YWTD β-propeller domain of an LRP5 or LRP6 receptor protein. In one specific embodiment, the polypeptide comprises an LRP5 and the in-frame deletion of base pairs is between nucleotide positions 2039-2466 of LRP5 mRNA. In an even more specific embodiment, the in-frame deletion is of 426 base pairs (2039-2466) of GenBank LRP5 accession no. AF064548. In another specific embodiment, the isolated nucleic acid molecule encodes a polypeptide comprising the amino acid sequence as set forth in SEQ ID NO: 5, and in yet a further specific embodiment, the isolated nucleic acid molecule encodes a polypeptide which comprises an amino acid sequence with greater than 70% homology with the amino acid sequence set forth in SEQ ID NO: 5 and activates a mammalian Wnt-signaling pathway. Most particularly, one isolated nucleic acid molecule embodiment encodes a polypeptide which comprises an amino acid sequence with greater than 90% homology with the amino acid sequence set forth in SEQ ID NO: 5 and activates a mammalian Wnt-signaling pathway.

The present invention further provides an embodiment directed to an isolated polypeptide comprising an LRP5 or LRP6 receptor having a mutation wherein the mutation comprises a deletion of a third YWTD β-propeller domain. According to one embodiment, the isolated polypeptide comprises a sequence of amino acids substantially identical to that set forth in SEQ ID NO:5.

The invention is further directed to an embodiment providing an isolated cell line. The isolated cell line comprises the nucleic acid molecule comprising a sequence of nucleotides having at least 90% homology with the sequence as set forth in SEQ ID NO: 1. The hall-mark of parathyroid cells is the unique expression of parathyroid hormone (PTH). The parathyroid cell line expresses parathyroid hormone and is obtained from parathyroid tumor cells according to methods described in the Examples, below.

The present invention is directed to several method embodiments. One such embodiment provides a method for diagnosing, prognosing, or determining the risk of developing an LRP5-related disease. LDL receptor-related protein 6 has 71% identity and is structurally similar to the protein encoded by the lrp5 gene, and the LRP5 and LRP6 receptor proteins share unique patterns of interaction and regulatory expression which distinguish them from the rest of the LDL receptor-related proteins. In addition, there is expectedly significant overlap in functionality and role in disease etiology and pathogenesis. Therefore, for purposes of defining the present invention, it should be understood that reference to an LRP5-related disease includes diseases in which a mutant LRP5 and/or LRP6 receptor is present. The method comprises: a) providing a tissue sample from a patient; b) detecting in the sample a mutant lrp5 gene or a mutant LRP5 receptor protein encoded by the mutant lrp5 gene; and c) relating presence of the mutant lrp5 gene or the mutant LRP5 receptor protein to an LRP5-related disease. In a specific embodiment, the detection step comprises PCR. The PCR comprises a step employing at least one forward and one reverse primer, selected from the group, consisting of,

(a) Forward:

(SEQ ID NO: 11)

5′-CTT CAC CAG CAG AGC CGC CAT CCA CAG-3′,
(b) Reverse:

(SEQ ID NO: 12)

5′-CCG GGA TCA TCC GAC TGA TG-3′,
(c) Forward:

(SEQ ID NO: 13)

5′-CAA GGC CAG CCG GGA CGT CA-3′,
and
(d) Reverse:

(SEQ ID NO: 14)

5′-AGG TAC CCT CGC TCC GCG TTG ACG ACG-3′;

and, an optional subsequent step employing at least one Nested Forward and one Reverse primer selected from the group consisting of,

(e) Nested Forward:

(SEQ ID NO: 15)

5′-GGA TCT CCC TCG AGA CCA ATA ACA ACG-3′,
(f) Nested Forward:

(SEQ ID NO: 16)

5′-CAT TGA CCA GCT GCC CGA CCT-3′,
(b) Reverse:
5′-CCG GGA TCA TCC GAC TGA TG-3′,
(d) Reverse:
5′-AGG TAC CCT CGC TCC GCG TTG ACG ACG-3′,

wherein if Forward primer (a) is employed in the step, then Nested Forward primer (e) is employed in the optional subsequent step, and if Forward primer (c) is employed in the step, then Nested Forward primer (f) is employed in the optional subsequent step, and wherein it is understood that a sequences (a)-(f) include the sequences complementary thereto. In other words, certain Nested primers may only be used with particular Forward primers such that in the optional step which employs a Nested primer one must take care to chose an appropriate such primer based upon which Forward primer was used initially. In an additional embodiment the PCR further comprises a step comprising detecting a mutant LRP5 PCR fragment by hybridization with a mutant LRP5-specific single stranded nucleic acid probe. The probe comprises a detectable signal, for example, the probe may be fluorescently labeled. In a very specific embodiment, the probe comprises a sequence as set forth in SEQ ID NO:17.

In another embodiment of this method, the detection step comprises analysis by gel electrophoresis, whereby a smaller mutant product is distinguishable from a larger non-mutant product. Gel electorphoresis is particularly suitable since it may be designed so that observed migration distance is a function of molecular size. Clearly in the present case the mutant lrp516 gene, which comprises a significant deletion when compared to the non-mutant, is therefore readily distinguishable.

In yet another embodiment of this method, the detection step comprises observing aberrant expression of at least one Wnt-signaling pathway target protein. In a specific embodiment, the at least one Wnt-signaling pathway target protein comprises β-catenin or c-myc, and in a very specific embodiment, the one Wnt-signaling pathway target protein comprises β-catenin.

In still another embodiment, the detection step comprises employing a ligand specific for the mutant LRP5 receptor and noting binding activity. One embodiment is directed to the method wherein the ligand comprises a peptide, protein or antibody and in a more specific embodiment the ligand comprises an antibody. In a very specific embodiment the ligand comprises a monoclonal antibody.

Another embodiment of the method provides that the LRP5-related disease comprises primary or secondary hyperparathyroidism, endocrine pancreatic tumor, breast, prostate, kidney, lung, thyroid, parathyroid or gastrointestinal tract carcinoma, or carcinoid tumor of the lung, thymus or gastrointestinal tract. In a more specific embodiment, the LRP5-related disease comprises primary or secondary hyperparathyroidism, parathyroid tumor, or breast carcinoma. In a very specific embodiment, the LRP5-related disease comprises primary or secondary hyperparathyroidism or parathyroid tumor.

The invention is further directed to an embodiment which provides a method of screening agents for an ability to modulate mutant LRP5 receptor activity. In this method, it is understood that LRP5 and LRP6 are analogous and the method steps should not be construed as limited to LRP5. The method comprises: a) generating a cell line which expresses a mutant LRP5 receptor; b) optionally, isolating the mutant LRP5 receptor from the cell line; c) providing a plurality of agents to be screened; d) providing a plurality of plates; e) plating each plate from the plurality of plates with at least one agent from the plurality of agents to be screened and either cells from a), or isolated mutant LRP5 receptors from b); f) incubating for a suitable period of time; and g) analyzing each plate from the plurality of plates to determine if the at least one agent modulates mutant LRP5 receptor activity. A suitable period of incubation is easily ascertainable by a person of ordinary skill in the art and may vary according to specific laboratory conditions, characteristics of the agents being screened, or other non-substantive intervening procedures/conditions. It is further apparent to a person of ordinary skill in the art that there will be instances where the order of plating according to step e) is material and instances where it is not and that this is also readily ascertainable. For purposes of this method, the analyzing step, step g), may comprise a step-wise procedure from an observed interaction to a conclusion that activity is modulated. Additionally, the ability to conduct the screening and analysis steps in a single plate makes this method particularly adaptable to high throughput screening, including automated screening, and may be employed to rapidly screen libraries of compounds. In one more specific embodiment, the cell line is the breast carcinoma cell line MCF7 (ATCC#IITB-22). In another specific embodiment, the cell line is a parathyroid cell line comprising the mutant lrp5 gene described herein, and/or expressing the mutant LRP5 receptor protein disclosed herein, and wherein the cell line expresses parathyroid hormone and is obtained from parathyroid tumor cells.

In an embodiment designed to eliminate from further consideration those agents which also modulate the non-mutant LRP5 receptor, the method further comprises screening the agent determined to modulate mutant LRP5 receptor activity for an ability to modulate non-mutant LRP5 activity by: a) providing a second cell line which does not express the mutant LRP5 and expresses a non-mutant LRP5 receptor; b) optionally, isolating the non-mutant LRP5 receptor from the cell line; c) proving at least one plate; d) plating the at least one plate with one or more agents determined to modulate mutant LRP5 receptor activity and one of either cells from a), or isolated non-mutant LRP5 receptors from b); e) incubating the at least one plate for a suitable period of time; and f) analyzing the at least one plate to determine if the one or more agents determined to modulate mutant LRP5 receptor activity also modulate non-mutant LRP5 receptor activity and identifying selected agents. It will be apparent to one of ordinary skill in the art that an agent may be selected either if it modulates activity of the mutant form but not the non-mutant form, or if the modulation is of the non-mutant receptor is desirable, neutral or not undesirable. It is contemplated that treatment methods directed to administering agents which modulate activity of the mutant receptor desirably do not adversely modulate activity of the non-mutant receptor. In a specific embodiment, the second cell line is HeLa (ATCC#CCL-2).

Another embodiment provides an additional step of testing the selected agent for efficacy in the suppression of LRP5-related diseases non-human animals. In specific embodiments, the screening method determines an agent which inhibits or inactivates mutant LRP5 receptor activity.

The design and use of small interfering RNAs (siRNA) complementary to mRNA targets that produce particular proteins is a tool employed to prevent translation of specific RNAs. SiRNAs have been shown to be capable of targeting specific RNA molecules in human cells. An siRNA is a segment of double stranded RNA that is from 15 to 30 nucleotides in length. It may be used to trigger a cellular reaction known as RNA interference. In RNA interference, double-stranded RNA is digested by an intracellular enzyme, producing siRNA duplexes. The siRNA duplexes bind to another intracellular enzyme complex, activating it to target whatever mRNA molecules are complementary to the siRNA sequence. The activated enzyme complex cleaves the targeted mRNA, destroying it and preventing it form being used to direct the synthesis of its corresponding protein product. Small interfering RNA vectors may be constructed by means well-known in the art to transfect humans.

The present inventors employ specific siRNAs to silence endogenous mutant LRP5 receptor expression in diseased parathyroid cells and abolish β-catenin accumulation. Accordingly, one specific embodiment of the method is directed to the ability to modulate mutant LRP5 receptor activity at a transcriptional level wherein the at least one agent is a small interfering RNA (siRNA). In a very specific embodiment, the siRNA comprises a sense RNA strand and an antisense RNA strand which form an RNA duplex, and the sense RNA strand comprises a nucleotide sequence substantially identical to a target sequence of about 18-25 contiguous nucleotides in mutant LRP5 mRNA. In an even more specific embodiment, the sense RNA strand comprises a nucleotide sequence as set forth in SEQ D NO: 9, and the antisense RNA strand comprises a nucleotide sequence as set forth in SEQ ID NO: 10.

Another embodiment of the present invention is directed to pharmaceutical composition comprising: at least one selected agent according to the methods for screening agents for an ability to modulate mutant LRP5 receptor activity, as described herein; and a pharmaceutically acceptable vehicle.

A further embodiment provides a method for reducing the production of at least one protein involved in the Wnt-signaling pathway mediated pathogenesis of tumors, comprising delivering an siRNA to the tumor. In one specific embodiment, the siRNA is delivered in the form of a viral vector comprising DNA encoding the siRNA.

An additional embodiment is directed to a method for identifying a ligand which modulates mutant LRP5 receptor activity. The method comprises: a) contacting a polypeptide comprising the amino acid sequence set forth as SEQ ID NO: 5, or a ligand-binding fragment thereof, with at least one ligand; and b) determining binding activity of the at least one ligand with respect to the polypeptide. In a specific embodiment, the polypeptide is expressed by a cell-line which has been transfected with a nucleic acid comprising a nucleic acid sequence which hybridizes with at least 90% homology to SEQ ID NO: 1. In a very specific embodiment, the cell line is obtained from mammalian tumor cells, and in a more specific embodiment, the cell line is obtained from mammalian parathyroid tumor cells. In an even more specific embodiment, the cell line is obtained from human parathyroid tumor cells and expresses parathyroid hormone. In one particular embodiment of the method, the nucleic acid sequence is substantially identical to that set forth in SEQ ID NO: 1.

An additional embodiment of the invention is directed to a method of determining the therapeutic effectiveness of a tumor/cancer treatment. The method comprises: a) providing tumor/cancer cells; b) determining mutant LRP5 receptor activity in the tumor/cancer cells; c) providing treated tumor/cancer cells; d) determining mutant LRP5 receptor activity in the treated tumor/cancer cells; e) comparing b) to d) wherein a decrease in mutant LRP5 receptor activity in d) relative to b) indicates the treatment is therapeutically effective. In one more specific embodiment, the receptor activity relates to overexpression of at least one Wnt-signaling pathway target protein. According to one embodiment, the Wnt-signaling pathway target protein is β-catenin or c-myc, and according to a more specific embodiment, the Wnt-signaling pathway target protein is β-catenin.

Also included within the scope of the present invention is an embodiment directed to a transgenic non-human animal having a genome comprising a having at least 90% homology with the sequence of nucleotides as set forth in SEQ ID NO: 1. Transfecting an organism with non-native genetic material may be accomplished by means well known and well established in the art.

A further embodiment is directed to a kit for diagnosing or prognosing a disease characterized by the expression of a mutant LRP5 or a mutant LRP6 receptor in a tissue. Such kits provide a rapid and on-site means for diagnosing and prognosing disease. The present inventors contemplate that “diagnosing” also includes assessing an individual's risk for developing diseases characterized by the expression of mutant LRP5 and/or 6 receptors. They further contemplate that “prognosing” a disease includes, inter alia, monitoring efficacy of a treatment regime. The kit comprises: a) one or more reagents having specificity for a mutant lrp5 gene or a mutant LRP5 receptor expressed therefrom, wherein the one or more reagents emits a detectable signal in the presence of the mutant lrp5 gene or the mutant LRP5 receptor expressed therefrom which is different from that emitted in the absence of the mutant lrp5 gene or the mutant LRP5 receptor expressed therefrom; b) means to deliver the one or more reagents to the tissue; and c) means suitable to detect the detectable signal. A person of ordinary skill in the art will be familiar with many well-known detectable signal-detection means combinations suitably employable, including but not limited to those based on fluorescence, radioisotopes, cytotoxicity, and the like.

In a specific embodiment of the kit, the mutant lrp5 gene comprises an in-frame deletion mutation of 426 base pairs (2039-2466) of the LRP5 DNA/mRNA identified by GenBank accession no. AF064548 or comprises a sequence of nucleotides having at least 90% homology with the sequence set forth in SEQ.ID.NO: 1. In another specific embodiment of the kit, the one or more reagents comprise at least one primer selected from the group consisting of:

Forward:

(SEQ ID NO: 11)

5′-CTT CAC CAG CAG AGC CGC CAT CCA CAG-3′,
Nested Forward:

(SEQ ID NO: 15)

5′-GGA TCT CCC TCG AGA CCA ATA ACA ACG-3′,
Reverse:

(SEQ ID NO: 12)

5′-CCG GGA TCA TCC GAC TGA TG-3′,
Forward:

(SEQ ID NO: 13

5′-CAA GGC CAG CCG GGA CGT CA-3′,
Nested Forward:

(SEQ ID NO: 16)

5′-CAT TGA CCA GCT GCC CGA CCT-3′,
and
Reverse:

(SEQ ID NO: 14)

5′-AGG TAC CCT CGC TCC GCG TTG ACG ACG-3′.

The following examples are for illustrative purposes and are intended to aid in understanding certain embodiments of the invention and in establishing enablement where pertinent. Hence, they should not be construed as defining or limiting the scope of the invention as otherwise disclosed herein.

### EXAMPLES

It is understood by a person of ordinary skill in the art that many of the specific methods described herein may be substituted for by other well-known methods without altering the substantive results or conclusions drawn therefrom. The examples presented herein successfully employ the following methodological protocols:

Tissue Specimens

Parathyroid adenomas and hyperplastic glands from patients with pHPT and sHPT respectively, and MEN1-associated parathyroid tumors are acquired from patients diagnosed and operated on in routine clinical practice. Tissues are intraoperatively snap-frozen. Normal parathyroid tissue is obtained from glands inadvertently removed in conjunction with thyroid surgery where autotransplantation was not required or as normal parathyroid gland biopsies in patients subjected to parathyroidectomy. Informed consent and approval of ethical committee is achieved.

Immunohistochemistry and Western Blotting

Frozen tissue sections (6 μm) are stained as described in Segersten, U. et al. “25-hydroxyvitamin D₃-1α-hydroxylase expression in normal and pathological parathyroid glands,” J. Clin. Endocrinol. Metab. 87, 2967-2972 (2002), incorporated herein by reference, using an anti-β-catenin goat polyclonal antibody (Santa Cruz Biotechnology INC., Santa Cruz, USA, #sc-1496). Control sections include use of primary antiserum pre-incubated with an excess of immunizing peptide (Santa Cruz, sc-1496P). Most specimens are also stained with a mouse monoclonal anti-β-catenin antibody (Santa Cruz, #sc-7963) and some specimens with an anti-active-β-catenin mouse monoclonal antibody (Upstate, Lake Placid, USA, #05-665), showing similar results (not shown). Protein extracts for Western blotting analysis are prepared from 10 consecutive frozen tissue sections (6 μm) in Cytobuster Protein Extract Reagent (Novagen Inc., Madison, Wis., USA) with Complete protease inhibitor cocktail (Roche Diagnostics GmbH, Mannheim, Germany).

Quantitative Real-Time RT-PCR

Total RNA is extracted with TriZol Reagent (Gibco BRL, Life Technologies Inc., Gaithersburg, USA) according to the manufacturer's instructions and the RNA is subsequently treated with RQ1 DNase I (Promega Corp., Madison, USA) or TURBO DNase (Ambion Inc., Austin, Tex., USA) and proteinase K. Reverse transcription of total RNA is performed with hexamer random primers using the First-Strand cDNA Synthesis kit (Amersham Pharmacia Biotech, Uppsala, Sweden) according to the manufacturer's instructions. The following mRNA-specific PCR primers and labeled probes (5′FAM-sequence-3′TAMRA) are used. For β-catenin, forward; AGC CTG TTC CCC TGA GGG TAT TTG, reverse; GAC TTG GGA GGT ATC CAC ATC CTC and probe; TGG CTA CTC AAG CTG ATT TGA TGG. For c-myc, forward; AAG ACT CCA GCG CCT TCT CTC CGT, reverse; TGG GCT GTG AGG AGG TTT GCT GTG, and probe; AGC GAC TCT GAG GAG GAA CAA GAA. For cyclin D1, forward; TTC CTC TCC AAA ATG CCA GAG GCG GAG, reverse; CAC TCT GGA GAG GAA GCG TGT GAG GCG and probe; GCC ACA GAT GTG AAG TTC ATT TCC. For GAPDH, forward; CCA CCA TGG AGA AGG CTG GGG CTC A, reverse; ATC ACG CCA CAG TTT CCC GGA GGG G and probe; CAA GCT TCC CGT TCT CAG CC. For 28S rRNA, the Ribosomal RNA Control Reagents (VIC probe) are used (Applied Biosystems, Foster City, Calif., USA). All PCR reactions are performed on ABI PRISM® 7700 Sequence Detection System using the TaqMan PCR core Reagent Kit (Applied Biosystems). Each cDNA sample is analyzed in triplicate. Standard curves for the expressed genes are established by amplifying a purified PCR fragment covering the sites for probes and primers. 28S rRNA is used as internal standard for the transient transfection experiments.

Detection of the LRP5 Deletion by PCR and Northern Blotting

DNA from tumors and normal parathyroid tissues are prepared by standard procedures including proteinase K treatment and phenol extraction. DNA from blood is prepared using the Wizard Genomic DNA Purification Kit (Promega Corp.). The quality of the DNA preparations is assured by PCR analysis for the presence of c-myc promoter DNA. DNA or cDNA is amplified by primary or nested PCR using mRNA-specific primers spanning positions 1992-2932 of LRP5 (GenBank accession no. AF064548). Forward primer; CTT CAC CAG CAG AGC CGC CAT CCA CAG, nested forward; GGA TCT CCC TCG AGA CCA ATA ACA ACG, and reverse; CCG GGA TCA TCC GAC TGA TG. PCR amplification comprises DNA or cDNA, 25 pmol of each primer, 0.2 mM dNTPs, 1×PCR buffer, 1.5 mM MgCl₂ and 0.25 U Platinum Taq DNA polymerase (Invitrogen Corporation). PCR conditions are: denaturation at 95° C. for 60 s, followed by 40 cycles of denaturation for 20 s, annealing at 58° C. for 20 s and extension at 72° C. for 90 s and a final extension at 72° C. for 7 min. An annealing temperature of 61° C. and 40 cycles are used for nested amplification. DNA sequence analysis of 4 mutant DNA and corresponding cDNA fragments as well as 2 wild type fragments are performed on ABI 373A using the ABI Prism Dye Terminator Cycle Sequencing Ready Reaction kit (Applied Biosystems). The fragments are cloned into pCRII-TOPO (Invitrogen Corporation) before sequencing. All fragments encode an open reading frame. Expression of non-mutant LRP5 is also detected by RT-PCR using primers spanning nucleotide positions 2133-2563 (not shown). Northern blotting is done according to the manufacturer (Ambion Inc.). The radiolabeled probe consists of the non-mutant LRP5 Xho I/Kpn I cDNA fragment.

Transfection

Plasmid LRP5×666-809 is constructed by replacing the Xho I/Kpn I fragment of pcDNA3.1/LRP5 (expressing LRP5) and pcDNA3.1/V5-His/LRP5 (expressing tagged LRP5) with a Xho I/Kpn I digested PCR fragment harbouring the deletion Δ666-809. HEK 293T cells (kind gift of Dr. Nateri) are transfected with CsCl purified plasmid DNA using Fugene 6 (Roche Diagnostics Scandinavia AB, Bromma, Sweden). HeLa cells are transfected using Polyfect (Quiagen Inc., Valencia, Calif., USA). RNA (see above), chromatin (see CHIP assay) or cytosolic protein extract is prepared 24 h post-transfection. Protein extracts are analyzed by western blotting with anti-V5-HRP antibody (Invitrogen AB, Stockholm, Sweden), anti-active-β-catenin mouse monoclonal antibody, anti-actin goat polyclonal antibody, and anti-β-tubulin rabbit polyclonal antibody (Santa Cruz Biotechnology). Transfection of control siRNA (Qiagen Operon, Cologne, Germany) or siRNA specific for LRP5Δ666-809 (sense strand: TAACAACGACCUCACCAUUdTdT; antisense strand: AAUGGUGAGGUCGUUGUUAdTdT; synthesized by Thermo Electron Corp., Ulm, Germany) is done with the cationic transfection reagent jetSI-ENDO according to the manufacturers recommendations (Polyplus-Transfection SAS, Illkirch, France).

Establishing the Parathyroid Cell Line

The parathyroid cell line is established as follows. Parathyroid tumor cells are taken and prepared from a patient suffering secondary hyperparathyroidism. The cells are dissociated and purified from the parathyroid tumor according to published procedures. Cells are counted and suspended in less than one cell per 100 ml. Growth medium (DMEM containing 10% FCS and complemented with glutamine, streptomycin and penicillin). 100 ml of cell suspension is cultured in 96-well microplates. Growth medium is continuously changed over 45 days. After 45 days, six cell colonies are observed and removed for further cultivation in 35 mm plates. One of six cultures is observed to survive after cultivation in growth medium supplemented with 10 mM lithium chloride. After four passages, cells are harvested and subjected to examination with Western (protein) blotting and fluorescent immunostaining using an antibody specific for parathyroid hormone. PTH is clearly detected and expressed in all cells. In addition, expression of PTH mRNA is detected using RT-PCR as described above. The original parathyroid tumor and the established sHPT cell line express the in-frame deleted LRP5 receptor gene.

ChIP Assay

Chromatin immunoprecipitation of transfected cells is performed using a protocol from Upstate, but with immunoprecipitation conditions as described by Chen et al. in “Regulation of hormone-induced histone hyperacetylation and gene activation of an acetylase,” Cell 98, 675-686 (1999). The anti-active-β-catenin mouse monoclonal antibody described by van Noort et al. in “Wnt-signaling controls the phosphorylation status of beta-catenin,” J. Biol. Chem. 277, 17901-17905 (2002), is used and c-myc promoter DNA, containing Tcf-4 binding site 2, is PCR amplified in the linear range by primers forward; ACG TGG CAA TGC GTT GCT GGG and reverse; ACA CAG AGA ACG CAC TGC GCG.

Statistical Analysis

An unpaired t test is used for all statistical analyses. Values herein are presented as arithmetical mean ±SEM. A p value of <0.05 is considered significant.

Example

(a) The following experiment illustrates that neither increased mRNA levels nor protein stabilizing mutations are plausible explanations for the β-catenin protein overexpression observed in all analyzed parathyroid tumors.

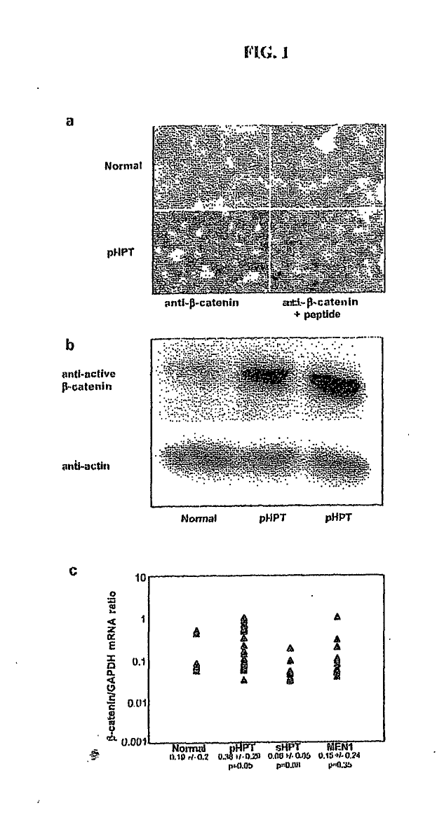

In FIG. 1a representative immunostainings of one normal parathyroid specimen and one parathyroid adenoma are shown. An anti-β-catenin goat polyclonal antibody is used as control and the antiserum is preabsorbed with an excess of immunizing peptide. All 63 analyzed parathyroid tumors show accumulation of β-catenin in comparison to normal tissue (n=6). FIG. 1b shows Western blotting of one normal parathyroid tissue specimen and two pHPT tumors. An anti-active-β-catenin monoclonal antibody is used. Overexposure is shown to reveal the weak β-catenin signal in the normal tissue. FIG. 1c shows determination of β-catenin/GAPDH mRNA expression ratio for 5 normal parathyroid gland specimens, 17 parathyroid adenomas of pHPT, 10 hyperplastic glands of sHPT, and 13 MEN1-associated parathyroid tumors by quantitative real-time RT-PCR. The ¹⁰log-transformed β-catenin/GAPDH ratio for each specimen and the arithmetical mean values ±SEM and P values for each tumor group are shown. A triangle represents the value for a single specimen. For some specimens the values overlap or partially overlap.

In contrast to normal parathyroid tissue (n=6), all analyzed parathyroid tumors from patients with primary HPT (pHPT; n=37), secondary HPT (sHPT; n=10), or HPT associated with the multiple endocrine neoplasia type 1 (MEN1) syndrome (n=16) demonstrate accumulation of β-catenin. All 63 parathyroid tumors show strongly increased, but somewhat variable staining for β-catenin, compared to the normal parathyroid specimens (FIG. 1a). Distinct accumulation of β-catenin is observed in the cells, and appears in the nucleus no more than in the cytoplasm. Western (protein) blotting reveals clearly increased β-catenin levels (FIG. 1b). The overall β-catenin mRNA expression levels of normal glands and the three tumor groups display small differences, with a considerable variation in mRNA level between individual specimens (FIG. 1c). Thus, no relation of β-catenin protein expression to β-catenin mRNA expression is demonstrated. The possibility of stabilizing β-catenin mutations of amino acid residues serine 33, serine 37, threonine 41, and serine 45 by DNA sequencing of ten adenomas from patients with pHPT is demonstrated. Similarly, no mutation of lysine 49, which is frequently mutated in anaplastic thyroid carcinoma, is detected.

(b) This portion of the experiment illustrates that the mutant LRP5/6 receptors activate β-catenin signaling in parathyroid tumors.

The LRP5 receptor gene is located at chromosome 11q13, a chromosomal region frequently associated with parathyroid tumor development. It is reasoned that genetic lesions in the LRP5 receptor might activate β-catenin signaling in parathyroid tumors, in particular since it is known that a truncation mutant of LRP5 lacking the extracellular domain is constitutively active in vitro. See Mao, J. et al. “Low-density lipoprotein receptor-related protein-5 binds to axin and regulates the canonical Wnt-signaling pathway,” Mol. Cell 7, 801-809 (2001).

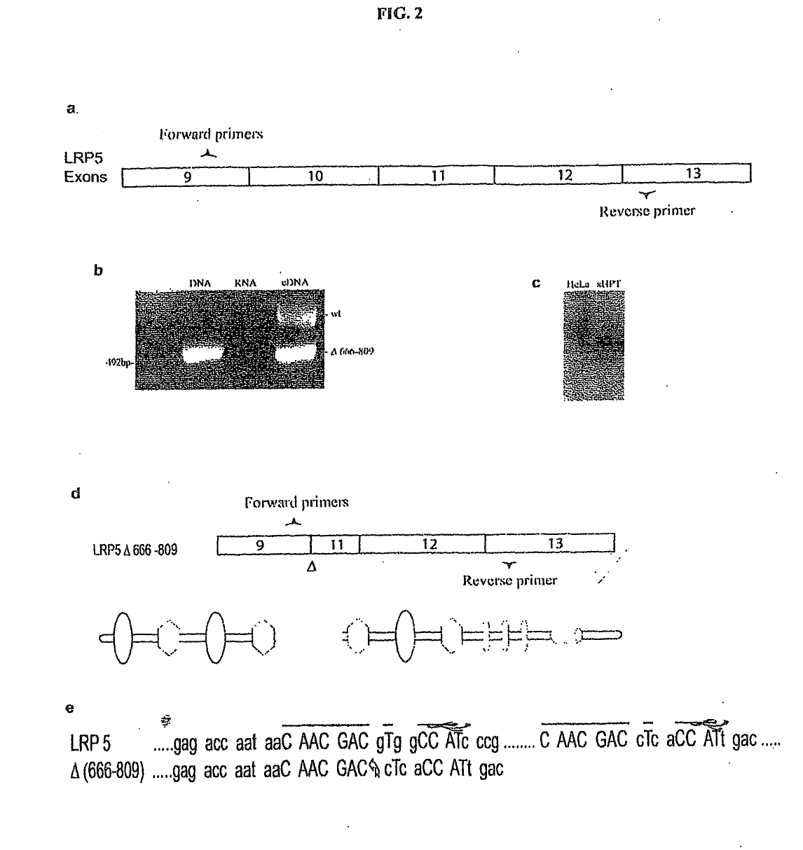

Using PCR with exon-specific primers (FIG. 2a), a deletion of the LRP5 tumor cDNA as well as DNA (FIG. 2b) is found in 20 out of all 23 analyzed parathyroid tumors (17 out of 20 pHPT tumors, 2 sHPT tumors and 1 MEN1 parathyroid tumor). Normal LRP5 mRNA is also expressed in the parathyroid tumors, including those with LRP5 deletion. (FIG. 2b). Normal LRP5 sequences without deletion are detected in four analyzed apparently normal parathyroid tissue specimens. The LRP5 deletion is not observed in constitutional DNA from blood in 4 analyzed HPT patients with tumor-associated mutation, nor in 21 patients with unrelated disease. As expected, Northern (RNA) blotting identifies a somewhat shorter LRP5 transcript in sHPT tumor cells compared to HeLa cells expressing non-mutant LRP5 (FIG. 2c). The in-frame deletion of 142 amino acids (Δ666-809), encompasses the third YWTD β-propeller domain between the second and third EGF repeats of LRP5 (FIG. 2d), see Jeon, H. et al “Implications for familial hypercholesterolemia from the structure of the LDL receptor YWTD-EGF domain pair,” Nat Struct Biol. 8, 499-504 (2001). The deleted LRP5 sequence is flanked by an imperfect direct repeat suggestive of some kind of illegitimate recombination (FIG. 2e).

FIG. 2 illustrates an in-frame deletion of LRP5 detected in parathyroid tumor DNA and cDNA. FIG. 2a shows the LRP5 mRNA-specific PCR primers used. The deletion is detected by primary PCR for most of the tumors or with nested PCR using an additional overlapping forward primer. FIG. 2b shows representative results from a PCR analysis using the primers shown in FIG. 2a, DNA, RNA, or cDNA from one pHPT tumor 123 bp DNA ladder in lane 1. FIG. 2c shows Northern blotting of RNA from HeLa cells expressing non-mutant LRP5 and a sHPT tumor cell line. FIG. 2d shows that the in-frame deletion of LRP5 between amino acids 666 and 809 encompasses the third YWTD β-propeller domain. A schematic structure of LRP5 is shown with YWTD β-propellers, epidermal growth factor-like repeats, low-density lipoprotein receptor-like ligand binding domains, and the transmembrane domain. FIG. 2e shows that the deleted part of LRP5 is flanked by a partial direct repeat. The Δ666-809 is between nucleotide positions 2039 and 2466 of the LRP5 mRNA (GenBank accession no. AF064548).

Thus, β-catenin protein is overexpressed in all parathyroid tumors and the LRP5 deletion is detected in 87% of these tumors. Mutation elsewhere in LRP5 or in other Wnt-signaling components leading to β-catenin accumulation in the remaining 13% of parathyroid tumors may be anticipated.

The results strongly indicate a critical role of activated Wnt-signaling through β-catenin in the etiology of primary, secondary, and MEN1-associated HPT. The fact that parathyroid tumors from patients with the familial MEN1 syndrome, where sequential inactivation of both copies of the MEN1 tumor suppressor gene may lead to uncontrolled cell growth (see Schussheim, D. H. et al. “Multiple endocrine neoplasia type 1: new clinical and basic findings,” Trends Endocrinol. Metab. 12, 173-178 (2001)), also display an aberration of the Wnt-signaling pathway, further suggests a fundamental role of β-catenin accumulation in neoplastic HPT. Other human tumors showing accumulation of β-catenin without mutation of Wnt-signaling components should be re-examined for potential mutations in LRP5 and/or LRP6.

The mRNA-specific PCR primers used herein identify the same mutated fragment in tumor DNA as in cDNA (FIG. 2b), i.e. without introns. This appears to represent the first example of an active retrogene-like DNA structure in tumors. Endogenous reverse transcriptase activity encoded by retrotransposons or endogenous retroviruses might be prominent in pathological parathyroid tissues as has been demonstrated in other tumor types. Presence of direct repeats in transcripts (FIG. 2e) might create specific deletions during the process of reverse transcription. It is possible that in other neoplasms where deregulated reverse transcriptase activity occurs, reverse transcribed mRNAs with mutations other than LRP5 might have been selected for.

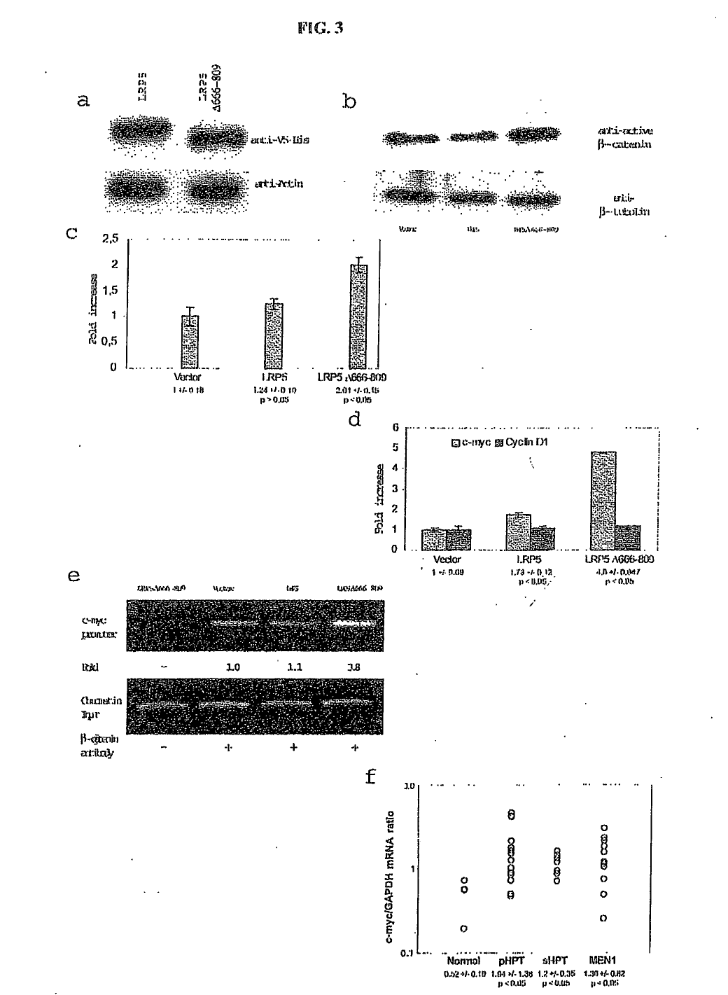

(c) In this experiment, the functional consequence of the LRP5 deletion (Δ666-809) by transfection of cells cultured in vitro is analyzed and compared to LRP5 and the results demonstrate that exogenous expression of LRP5Δ666-809 at similar protein expression levels results in a higher level of stabilized β-catenin (FIG. 3a,b).

FIGS. 3a-3f illustrate β-catenin accumulation and target gene transcription in mutant LRP5 expressing cells. FIG. 3a shows Western blotting of transiently expressed V5-tagged LRP5 and LRP5Δ666-809 in HEK293T cells. FIG. 3b shows cytosolic fractions of transiently transfected cells analyzed for non-phosphorylated (active) β-catenin protein expression. FIG. 3c shows Topflash reporter gene activity in transiently transfected HeLa cells. FIG. 3d shows β-catenin target gene expression in transfected cells, quantified by real-time RT-PCR. The values for c-myc expression are shown as well. FIG. 3e shows chromatin immunoprecipitation of the c-myc promoter in transfected cells. An anti-active-β-catenin monoclonal antibody is used. FIG. 3f shows c-myc mRNA overexpression in parathyroid tumors. The c-myc/GAPDH mRNA expression ratios are determined by quantitative real-time RT-PCR in the same parathyroid specimens as described in the legend to FIG. 1c. An open circle represents the value for a single specimen. For some specimens the values overlap or partially overlap.

The Topflash luciferase reporter construct, which carries a minimal promoter with TCF-binding sites which is activated by the TCF/β-catenin complex and as described by Korinek, V. et al. in “Constitutive transcriptional activation by a beta-catenin-Tcf complex in APC −/− colon carcinoma,” Science 275, 1784-1787 (1997), is employed to see whether the augmented β-catenin level also results in enhanced transcription. A significant modest 2-fold effect is seen with LRP5Δ666-809 (FIG. 3c).

Whether LRP5Δ666-809 could affect endogenously expressed β-catenin target genes is tested by determining the cyclin D1 and c-myc mRNA levels from transfection experiments. LRP5Δ666-809 causes a five-fold increase of c-myc mRNA level while cyclin D1 is unaffected, compared to LRP5 in transfected cells (FIG. 3d). No additional effects are seen with co-transfected Wnt-1 (data not shown). These results indicate constitutive activation of c-myc gene transcription by the in-frame deletion mutant LRP5Δ666-809. This is further supported by the simultaneously enhanced association of β-catenin to the c-myc promoter (4-fold), as revealed by chromatin immunoprecipitation (FIG. 3e).

(d) This experiment is designed to assess the relevance of the observations from cell culture experiments by relating the c-myc mRNA expression level of the various parathyroid tumors to that of normal parathyroid tissue.

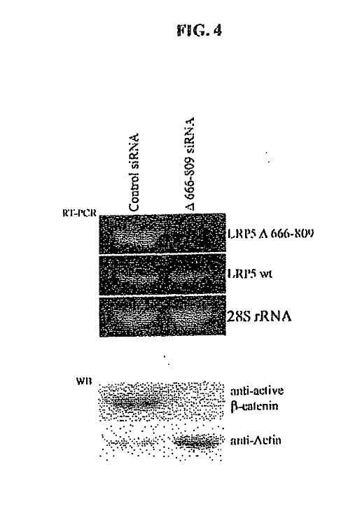

C-myc mRNA expression is significantly higher in the parathyroid adenomas, secondary hyperplastic glands, and in the MEN1-associated parathyroid tumors as compared to the normal tissue specimens (FIG. 3f). β-catenin protein accumulation in connection with increased c-myc mRNA expression is not observed for all individual tumor specimens. Finally, down-regulation of endogenous LRP5Δ666-809 expression by small interfering RNA in the sHPT parathyroid cell line (FIG. 2c) leads to abolished accumulation of β-catenin (FIG. 4).

The foregoing examples are intended to be illustrative of certain embodiments of the present invention and the scope of the invention should not be construed as limited in any way by the examples disclosed herein.

1. An isolated nucleic acid molecule having at least 90% homology with the sequence of nucleotides as set forth in SEQ ID NO: 1.

2. An isolated nucleic acid molecule comprising a sequence of nucleotides substantially identical to that set forth in SEQ ID NO: 1.

3. An isolated nucleic acid molecule encoding a polypeptide comprising a mutant low density lipoprotein related protein 5 (LRP5) or 6 (LRP6), the molecule comprising an in-frame deletion of base pairs encoding a third YWTD β-propeller domain of an LRP5 or LRP6 receptor protein.

4. The isolated nucleic molecule as recited in claim 3 wherein the polypeptide comprises a LRP5 and the in-frame deletion of base pairs is between nucleotide positions 2039-2466 of LRP5 mRNA.

5. The isolated nucleic acid molecule as recited in claim 4, wherein the in-frame deletion is of 426 base pairs (2039-2466) of GenBank LRP5 accession no. AF064548.

6. The isolated nucleic acid molecule as recited in claim 5 encoding a polypeptide comprising the amino acid sequence as set forth in SEQ ID NO: 5.

7. The isolated nucleic acid molecule as recited in claim 3, encoding a polypeptide which comprises an amino acid sequence with greater than 70% homology with the amino acid sequence set forth in SEQ ID NO: 5 and activates a mammalian Wnt-signaling pathway.

8. The isolated nucleic acid molecule as recited in claim 3, encoding a polypeptide which comprises an amino acid sequence with greater than 90% homology with the amino acid sequence set forth in SEQ ID NO: 5 and activates a mammalian Wnt-signaling pathway.

9. An isolated polypeptide comprising an LRP5 or LRP6 receptor having a mutation wherein the mutation comprises a deletion of a third YWTD β-propeller domain.

10. The isolated polypeptide as recited in claim 9 comprising a sequence of amino acids substantially identical to that set forth in SEQ ID NO:5.

11. An isolated cell line comprising a nucleic acid molecule having at least 90% homology with the sequence of nucleotides as set forth in SEQ ID NO: 1, or comprising a nucleic acid molecule which expresses a polypeptide having an amino acid sequence with greater than 70% homology to the amino acid sequence set forth in SEQ ID NO: 5, wherein the cell line expresses parathyroid hormone and is obtained from parathyroid tumor cells.

12. A method for diagnosing, prognosing, or determining the risk of developing an LRP5-related disease comprising:
a) providing a tissue sample from a patient;
b) detecting in the sample a mutant lrp5 gene or a mutant LRP5 receptor protein encoded by the mutant lrp5 gene; and
c) relating presence of the mutant lrp5 gene or the mutant LRP5 receptor protein to an LRP5-related disease.

13. The method as recited in claim 12 wherein the detection step comprises PCR comprising: a step employing at least one forward and one reverse primer, selected from the group, consisting of,
(a) Forward:
(SEQ ID NO: 11)
5′-CTT CAC CAG CAG AGC CGC CAT CCA CAG-3′,
(b) Reverse:
(SEQ ID NO: 12)
5′-CCG GGA TCA TCC GAC TGA TG-3′,
(c) Forward:
(SEQ ID NO: 13)
5′-CAA GGC CAG CCG GGA CGT CA-3′,
and
(d) Reverse:
(SEQ ID NO: 14)
5′-AGG TAC CCT CGC TCC GCG TTG ACG ACG-3′;
and, an optional subsequent step employing at least one Nested Forward and one Reverse primer selected from the group consisting of,
(e) Nested Forward:
(SEQ ID NO 15)
5′-GGA TCT CCC TCG AGA CCA ATA ACA ACG-3′,
(f) Nested Forward:
(SEQ ID NO: 16)
5′-CAT TGA CCA GCT GCC CGA CCT-3′,
(b) Reverse:
(SEQ ID NO: 12)
5′-CCG GGA TCA TCC GAC TGA TG-3′,
(d) Reverse:
(SEQ ID NO: 14)
5′-AGG TAC CCT CGC TCC GCG TTG ACG ACG-3′,
wherein if Forward primer (a) is employed in the step, then Nested Forward primer (e) is employed in the optional subsequent step, and if Forward primer (c) is employed in the step, then Nested Forward primer (f) is employed in the optional subsequent step, and wherein a sequence complementary to a sequence (a)-(f) may be employed in place of the sequence (a)-(f).

14. The method as recited in claim 13 further comprising a second subsequent optional step comprising detecting a mutant LRP5 PCR fragment by hybridization with a detectable mutant LRP5-specific single stranded nucleic acid probe.

15. The method as recited in claim 14 wherein the detectable mutant LRP5-specific single stranded nucleic acid probe is fluorescently labeled.

16. The method as recited in claim 15 wherein the detectable mutant LRP5-specific single stranded nucleic acid probe comprises a nucleic acid sequence as set forth in SEQ ID NO: 17.

17. The method as recited in claim 12 wherein the detection step comprises analysis by gel electrophoresis, whereby a smaller mutant product is distinguishable from a larger non-mutant product.

18. The method as recited in claim 12 wherein the LRP5-related disease is a disease in which a mutant LRP5 and/or LRP6 receptor is present.

19. The method as recited in claim 12 wherein the detection step comprises observing aberrant expression of at least one Wnt-signaling pathway target protein.

20. The method as recited in claim 19 wherein the at least one Wnt-signaling pathway target protein comprises β-catenin.

21. The method as recited in claim 12 wherein the detection step comprises employing a ligand specific for the mutant LRP5 receptor and noting binding activity.

22. The method as recited in claim 21 wherein the ligand comprises a peptide, protein or antibody.

23. The method as recited in claim 21 wherein the ligand comprises an antibody.

24. The method as recited in claim 21 wherein the ligand comprises a monoclonal antibody.

25. The method as recited in claim 12 wherein the LRP5-related disease comprises primary or secondary hyperparathyroidism, endocrine pancreatic tumor, breast, prostate, kidney, lung, thyroid, parathyroid or gastrointestinal tract carcinoma, or carcinoid tumor of the lung, thymus or gastrointestinal tract.

26. The method as recited in claim 12 wherein the LRP5-related disease comprises primary or secondary hyperparathyroidism or parathyroid tumor.

27. A method of screening a plurality of agents for an ability to modulate mutant LRP5 receptor activity, the method comprising:
a) generating a cell line which expresses a mutant LRP5 receptor;
b) optionally, isolating the mutant LRP5 receptor from the cell line;
c) providing a plurality of agents to be screened;
d) providing a plurality of plates
e) plating each plate from the plurality of plates with at least one agent from the plurality of agents to be screened and either cells from a), or isolated mutant LRP5 receptors from b);
f) incubating for a suitable period of time; and
g) analyzing each plate from the plurality of plates to determine if the at least one agent modulates mutant LRP5 receptor activity.

28. The method as recited in claim 27 wherein the cell line is the breast carcinoma cell line MCF7 (ATCC#IITB-22).

29. The method as recited in claim 27 wherein the cell line is the parathyroid cell line recited according to claim 11.

30. The method as recited in claim 27 further comprising screening an agent determined to modulate mutant LRP5 receptor activity for an ability to modulate non-mutant LRP5 activity by:
a) providing a second cell line which does not express the mutant LRP5 and expresses a non-mutant LRP5 receptor;
b) optionally, isolating the non-mutant LRP5 receptor from the cell line;
c) proving at least one plate;
d) plating the at least one plate with one or more agents determined to modulate mutant LRP5 receptor activity and one of either cells from a), or isolated non-mutant LRP5 receptors from b);
e) incubating the at least one plate for a suitable period of time; and
f) analyzing the at least one plate to determine if the one or more agents modulate non-mutant LRP5 receptor activity and identifying any remaining agent as a selected agent.

31. The method as recited in claim 30 wherein the second cell line is HeLa (ATCC#CCL-2).

32. The method as recited in claim 30 further comprising testing the selected agent for efficacy in the suppression of LRP5-related diseases non-human animals.

33. The method according to claim 27 wherein the ability to modulate mutant LRP5 receptor activity is at a transcriptional level and the at least one agent is a small interfering RNA (siRNA).

34. The method as recited in claim 33 wherein the siRNA comprises a sense RNA strand and an antisense RNA strand which form an RNA duplex, and wherein the sense RNA strand comprises a nucleotide sequence substantially identical to a target sequence of about 18-25 contiguous nucleotides in mutant LRP5 mRNA.

35. The method as recited in claim 34 wherein the sense RNA strand comprises a nucleotide sequence as set forth in SEQ ID NO: 9, and the antisense RNA strand comprises a nucleotide sequence as set forth in SEQ ID NO: 10.

36. A pharmaceutical composition comprising: at least one selected agent according to the methods recited in any of claims 30-35; and a pharmaceutically acceptable vehicle.

37. A method for reducing the production of at least one protein involved in the Wnt-signaling pathway mediated pathogenesis of tumors, comprising delivering an siRNA to the tumor.

38. The method as recited in claim 37 wherein the siRNA is delivered in the form of a viral vector comprising DNA encoding the siRNA.

39. A screening method as recited in claim 27 wherein the screening method determines an agent which inhibits or inactivates mutant LRP5 receptor activity.

40. A method for identifying a ligand which modulates mutant LRP5 receptor activity, the method comprising:
a) contacting a polypeptide comprising the amino acid sequence set forth as SEQ ID NO: 5, or a ligand-binding fragment thereof, with at least one ligand; and
b) determining binding activity of the at least one ligand with respect to the polypeptide.

41. The method as recited in claim 40 wherein the polypeptide is expressed by a cell-line which has been transfected with a nucleic acid comprising a nucleic acid sequence which hybridizes with at least 90% homology to SEQ ID NO: 1.

42. The method as recited in claim 41 wherein the cell line is obtained from mammalian tumor cells.

43. The method as recited in claim 41 wherein the cell line is obtained from mammalian parathyroid tumor cells.

44. The method as recited in claim 41 wherein the cell line is obtained from human parathyroid tumor cells and expresses parathyroid hormone.

45. The method as recited in claim 41 wherein the nucleic acid sequence is substantially identical to that set forth in SEQ ID NO: 1.

46. A method of determining the therapeutic effectiveness of a tumor/cancer treatment comprising:
a) providing tumor/cancer cells;
b) determining mutant LRP5 receptor activity in the tumor/cancer cells;
c) providing treated tumor/cancer cells;
d) determining mutant LRP5 receptor activity in the treated tumor/cancer cells;
e) comparing b) to d) wherein a decrease in mutant LRP5 receptor activity in d) relative to b) indicates the treatment is therapeutically effective.

47. The method as recited in claim 46 wherein the receptor activity relates to overexpression of at least one Wnt-signaling pathway target protein.

48. The method as recited in claim 47 wherein the Wnt-signaling pathway target protein is β-catenin or c-myc.

49. The method as recited in claim 46 wherein the Wnt-signaling pathway target protein is β-catenin.

50. A transgenic non-human animal having a genome comprising a having at least 90% homology with the sequence of nucleotides as set forth in SEQ ID NO: 1.

51. A kit for diagnosing or prognosing a disease characterized by the expression of a mutant LRP5 receptor in a tissue, comprising:
a) one or more reagents having specificity for a mutant lrp5 gene or a mutant LRP5 receptor expressed therefrom, wherein the one or more reagents emits a detectable signal in the presence of the mutant lrp5 gene or the mutant LRP5 receptor expressed therefrom which is different from a signal emitted in the absence of the mutant lrp5 gene or the mutant LRP5 receptor expressed therefrom;
b) means to deliver the one or more reagents to the tissue; and

52. The kit as recited in claim 51 wherein the mutant lrp5 gene comprises an in-frame deletion mutation of 426 base pairs (2039-2466) of the LRP5 DNA/mRNA identified by GenBank accession no. AF064548.

53. The kit as recited in claim 51 wherein the one or more reagents comprise at least one primer selected from the group consisting of:
Forward:
5′-CTT CAC CAG CAG AGC CGC CAT CCA CAG-3′,
Nested Forward:
5′-GGA TCT CCC TCG AGA CCA ATA ACA ACG-3′,
Reverse:
5′-CCG GGA TCA TCC GAC TGA TG-3′,
Forward:
5′-CAA GGC CAG CCG GGA CGT CA-3′,
Nested Forward:
5′-CAT TGA CCA GCT GCC CGA CCT-3′,
and
Reverse:
5′-AGG TAC CCT CGC TCC GCG TTG ACG ACG-3′.


SEQUENCE LISTING (17 sequences)

SEQ ID NO: 1 - DNA, 737 bp, homo sapiens
caaggccagccgggacgtcatcattgaccagctgcccgacctgatggggctcaaagctgtgaatgtggccaaggtcgtcggaaccaacccgtgtgcggacaggaacggggggtgcagccacctgtgcttcttcacaccccacgcaacccggtgtggctgccccatcggcctggagctgctgagtgacatgaagacctgcatcgtgcctgaggccttcttggtcttcaccagcagagccgccatccacaggatctccctcgagaccaataacaacgacctcaccattgactacgctgaccagcgcctctactggaccgacctggacaccaacatgatcgagtcgtccaacatgctgggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcctggtgttccactcctcccgccaggatggcctcaatgactgtatgcacaacaacgggcagtgtgggcagctgtgccttgccatccccggcggccaccgctgcggctgcgcctcacactacaccctggaccccagcagccgcaactgcagcccgcccaccaccttcttgctgttcagccagaaatctgccatcagtcggatgatcccgg

SEQ ID NO: 2-4 - skipped (3 entries)

SEQ ID NO: 5 - protein, 245 aa, homo sapiens
KASRDVIIDQLPDLMGLKAVNVAKVVGTNPCADRNGGCSHLCFFTPHATRCGCPIGLELLSDMKTCIVPEAFLVFTSRAAIGRISLETNNNDLTIDYADQRLYWTDLDTNMIESSNMLGQERVVIADDLPHPFGLTQYSDYIYWTDWNLHSIERADKTSGRNRTLIQGHLDFVMDILVFHSSRQDGLNDCMHNNGQCGQLCLAIPGGHRCGCASHYTLDPSSRNCSPPTTFLLFSQKSAISRMIP

SEQ ID NO: 6-8 - skipped (3 entries)

SEQ ID NO: 9 - DNA, 23 bp, artificial
  siRNA; sense strand
taacaacgaccucaccauudtdt

SEQ ID NO: 10 - DNA, 23 bp, Artificial
  siRNA; antisense strand
aauggugaggucguuguuadtdt

SEQ ID NO: 11 - DNA, 27 bp, Artificial
  forward primer
cttcaccagcagagccgccatccacag

SEQ ID NO: 12 - DNA, 20 bp, Artificial
  reverse primer
ccgggatcatccgactgatg

SEQ ID NO: 13 - DNA, 20 bp, Artificial
  forward primer
caaggccagccgggacgtca

SEQ ID NO: 14 - DNA, 27 bp, Artificial
  reverse primer
aggtaccctcgctccgcgttgacgacg

SEQ ID NO: 15 - DNA, 27 bp, Artificial
  nested forward primer
ggatctccctcgagaccaataacaacg

SEQ ID NO: 16 - DNA, 21 bp, Artificial
  nested forward primer
cattgaccagctgcccgacct

SEQ ID NO: 17 - DNA, 21 bp, Artificial
  mutant LRP5 probe
aacaacgacctcaccattgac